(19) United States
(12) Patent Application Publication
(10) Pub. No.: US 2014/0221741 A1
(43) Pub. Date: Aug. 7, 2014

(54) Self Assembly of In-Vivo Capsule System

(71) Applicant: CAPSO VISION, INC., Saratoga, CA (US)

(72) Inventors: Kang-Huai Wang, Saratoga, CA (US); Gordon C. Wilson, San Francisco, CA (US)

(73) Assignee: CAPSO VISION, INC., Saratoga, CA (US)

(21) Appl. No.: 13/762,153

(22) Filed: Feb. 7, 2013

(51) Int. Cl. A61B 1/00

(52) U.S. Cl. CPC A61B 1/00064; A61B 1/00016
USPC 600/109; 600/101; 600/160

(57) ABSTRACT

An in-vivo self-assembly capsule system is disclosed, where the in-vivo capsule system comprises a first primary capsule, at least one second capsule, and a self-connection means for connecting the first primary capsule and said at least one second capsule by providing holding force when the first primary capsule and said at least one second capsule are in contact in human GI tract. The self-connection means comprises an interlocking means disposed on the first primary capsule and said at least one second capsule. The interlocking means may correspond to hooks disposed on one connecting side and loops disposed on another connecting side, or hooks disposed on both connecting sides. The interlocking means may also correspond to a first magnet and an interaction piece affixed to two corresponding capsules respectively, and the interaction piece corresponds to a second magnet or a ferromagnetic component.

### FIELD OF THE INVENTION

The present invention relates to in-vivo capsule system comprising multiple capsules that can assemble by itself within the human GI tract.

### BACKGROUND AND RELATED ART

Devices for imaging body cavities or passages in vivo are known in the art and include endoscopes and autonomous encapsulated cameras. Endoscopes are flexible or rigid tubes that pass into the body through an orifice or surgical opening, typically into the esophagus via the mouth or into the colon via the rectum. An image is formed at the distal end using a lens and transmitted to the proximal end, outside the body, either by a lens-relay system or by a coherent fiber-optic bundle. A conceptually similar instrument might record an image electronically at the distal end, for example using a CCD or CMOS array, and transfer the image data as an electrical signal to the proximal end through a cable. Endoscopes allow a physician control over the field of view and are well-accepted diagnostic tools. However, they do have a number of limitations, present risks to the patient, are invasive and uncomfortable for the patient, and their cost restricts their application as routine health-screening tools.

Capsule endoscopy is another established method for imaging the small bowel. In some market countries recently-introduced commercially-available capsules permit imaging of the colon as well. However, colon imaging presents a number of challenges that these capsules do not completely overcome. Patients cannot easily follow the protocols necessary to keep the colon clear of image-obstructing bile and fecal matter for longer than about 12 hours after swallowing the capsule. Thus, the capsule transit time should be less than 12 hours for the vast majority of patients, which, given the distribution of transit times among patients, requires a mean transit time of about 6-8 hours. Also, the capsule orientation is uncontrolled in the colon and mucosal surfaces may be hidden behind haustral folds. Existing colon capsules include a camera at both ends to increase the likelihood of visualizing regions of interest under these conditions. The capsule may sporadically move quickly through the colon. Thus, a high peak-frame-rate (e.g. 17 fps) is necessary to image all mucosal surfaces. In general, capsule endoscopy's effectiveness in the colon will benefit greatly from increased functionality and performance. Moreover, capsules of the future may include new functions such as propulsion, biopsy capture, and therapy delivery. The need to contain all of the hardware, including the power source (batteries), necessary to perform all system functions in a capsule sufficiently small to swallow limits the functionality and performance of capsule endoscopes. Additionally, an ingestible in vivo imaging system that is too large to swallow could move through the bowel more quickly with peristalsis and with a more stable orientation than one small enough to swallow. It is desirable to develop new capsule systems to support the increasing demand for capsule functionality and performance. Also, it is desirable to offer capsules with stable orientation while maintaining the capsule small enough to swallow easily.

### BRIEF SUMMARY OF THE INVENTION

An in-vivo self-assembly capsule system and a method for the manufacture of the in-vivo self-assembly capsule system are disclosed, where the in-vivo capsule system comprises a first primary capsule, at least one second capsule, and a self-connection means for connecting the first primary capsule and said at least one second capsule by providing holding force when the first primary capsule and said at least one second capsule are in contact in human GI tract. The second capsule may be a primary capsules or a secondary capsule. The self-connection means comprises an interlocking means disposed on the first primary capsule and said at least one second capsule.

The interlocking means may correspond to hooks disposed on one connecting side and loops disposed on the other connecting side, or hooks disposed on both connecting sides. At least a part of the first primary capsule or said at least one second capsule is coated with a coating material that will dissolve in the GI tract. The first primary capsule may have a first specific gravity larger than one and the secondary capsule may have a second specific gravity smaller than one. Therefore, when the primary capsule connects to the secondary capsule, the capsule system can maintain its desirable orientation when it travels through the GI tract. In order to help the primary capsule to connect to the secondary capsule, a dense material may be attached to the secondary capsule using a dissolvable shell.

The interlocking means may also correspond to a first magnet and an interaction piece affixed to two corresponding capsules respectively, and the interaction piece may correspond to a second magnet, a ferromagnetic component, or a ferrimagnetic component. The first magnet and the interaction piece can be disposed inside or outside the two corresponding capsules. Also, the first magnet and the interaction piece can be configured to penetrate through housings of the two corresponding capsules while maintaining the housings of the two corresponding capsules sealed. A pair of complimentary coded magnets can be used to cause the connection in a desired orientation. The coded magnets may also be properly configured to ensure that the attraction force is within a safe level if the separation between two coded magnets is above a threshold. In another embodiment, the first magnet and the interaction piece are formed into mating concave-convex contact surfaces to reduce the pressure on the tissue exerted by the capsules due to attraction force. Furthermore, an electrical connection can also be provided between the capsules.

The primary capsule may comprise an image sensor, an optical subsystem to form image onto the image sensor, light sources, and on-board storage to store captured images or a wireless module to transmit captured images. The capsule system may include two primary capsules, one primary capsule and one secondary capsule, or one primary capsule and two secondary capsules. When one primary capsule and two secondary capsules are used, the hooks/loops can be disposed on the first primary capsule and first ends of said two secondary capsules, and the loops/hooks can be disposed on second ends of said two secondary capsules. Alternatively, the hooks/loops can be disposed on the first primary capsule and one of said two secondary capsules, and the loops/hooks can be disposed on the other of said two secondary capsules.

### BRIEF DESCRIPTION OF THE DRAWINGS

FIG. 1A illustrates an exemplary in-vivo capsule system incorporating an embodiment of the present invention, where the in-vivo capsule system comprises two capsules.

FIG. 1B illustrates the two capsules of FIG. 1A in a separated state within the stomach.

FIG. 1C illustrates the two capsules of FIG. 1A in a connected state within the stomach.

FIG. 2A illustrates an exemplary in-vivo capsule system comprising a primary capsule and two secondary capsules, where the primary capsule includes a panoramic camera and the capsules are shown in a separated state.

FIG. 2B illustrates the capsules of FIG. 2A in a connected state.

FIG. 2C illustrates an exemplary in-vivo capsule system comprising a primary capsule and one secondary capsule, where the primary capsule includes a forward-looking camera and the capsules are shown in a separated state.

FIG. 2D illustrates the modules of FIG. 2C in a connected state.

FIG. 3 illustrates an exemplary in-vivo capsule system comprising two primary capsules, where each capsule has a single camera facing one end of the capsule.

FIG. 4 illustrates an exemplary in-vivo capsule system comprising one primary capsule and multiple secondary capsules with hook-and-loop connectors.

FIG. 5A-FIG. 5D illustrate four examples of capsule modules connected by attraction of magnets.

FIG. 6 illustrates an exemplary scenario that magnets attract through an intervening layer of tissue that may cause the tissue to become pinched and damaged.

FIG. 7A-FIG. 7B illustrate examples of coded magnets with complementary polarities.

FIG. 8A illustrates exemplary plots of the attraction force between magnetic connectors as a function of their axial separation, where the solid line is for two coded magnets, and the dashed line is for two standard magnets with uniform magnetization.

FIG. 8B illustrates an example of two magnet connectors of two respective modules with an intervening layer of tissues of thickness X, where the attraction force is at a safe level that will not damage or compress the tissue at separation X and X is the minimum tissue.

FIG. 9 illustrates an example that two capsules have magnetic connectors with domed surfaces facing the ends, and the magnets are connected and have a relative tilt of θ.

FIG. 10 illustrates an example that capsule module A has a flexible member between the main housing and connector A, and capsule module B has a connector that does not pivot.

FIG. 11A illustrates an example that one capsule has a convex surface and the other capsule has a concave surface at its tip.

FIG. 11B illustrates a scenario that the two capsules of FIG. 11A have an intervening layer of tissue.

FIG. 12 illustrates an example of an in-vivo capsule system that has an electrical connection between two capsules.

FIG. 13A shows an example of an in-vivo capsule system with a primary capsule of specific gravity greater than one and a secondary capsule with specific gravity less than one in the stomach.

FIG. 13B shows an in-vivo capsule system in a connected state, where the ensemble is balanced with the center of mass near the center of the assembly.

FIG. 14A-FIG. 14B illustrate various stages of an in-vivo capsule system incorporating an embodiment of the present invention; FIG. 14A illustrates the primary capsule and the secondary capsule prior to swallowing or just after swallowing; FIG. 14B illustrates that the coating on the primary capsule and the secondary capsule dissolves and the hooks and loops are exposed for connection; and FIG. 14C illustrates that a shell on the secondary capsule dissolves and the dense material becomes separated from the secondary capsule.

FIG. 15A illustrates an exemplary in-vivo capsule system having a primary capsule with hooks, a type-i secondary capsule with loops on two poles, and a type-ii secondary capsule with hooks around.

FIG. 15B illustrates an exemplary capsule system having a primary capsule with hooks and two type-iii secondary capsules with loops and hooks on two poles.

FIG. 16A illustrates an exemplary operation flow for an in-vivo capsule system incorporating an embodiment of the present invention, where the primary capsule includes on-board storage to store the captured images.

FIG. 16B illustrates an exemplary operation flow for an in-vivo capsule system incorporating an embodiment of the present invention, where the primary capsule includes a wireless module to transmit the captured images.

### DETAILED DESCRIPTION OF THE INVENTION

It will be readily understood that the components of the present invention, as generally described and illustrated in the figures herein, may be arranged and designed in a wide variety of different configurations. Thus, the following more detailed description of the embodiments of the systems and methods of the present invention, as represented in the figures, is not intended to limit the scope of the invention, as claimed, but is merely a representative of selected embodiments of the invention. References throughout this specification to “one embodiment,” “an embodiment,” or similar language mean that a particular feature, structure, or characteristic described in connection with the embodiment may be included in at least one embodiment of the present invention. Thus, appearances of the phrases “in one embodiment” or “in an embodiment” in various places throughout this specification are not necessarily all referring to the same embodiment.

Furthermore, the described features, structures, or characteristics may be combined in any suitable manner in one or more embodiments. One skilled in the relevant art will recognize, however, that the invention can be practiced without one or more of the specific details, or with other methods, components, etc. In other instances, well-known structures, or operations are not shown or described in detail to avoid obscuring aspects of the invention. The illustrated embodiments of the invention will be best understood by reference to the drawings, wherein like parts are designated by like numerals throughout. The following description is intended only by way of example, and simply illustrates certain selected embodiments of apparatus and methods that are consistent with the invention as claimed herein.

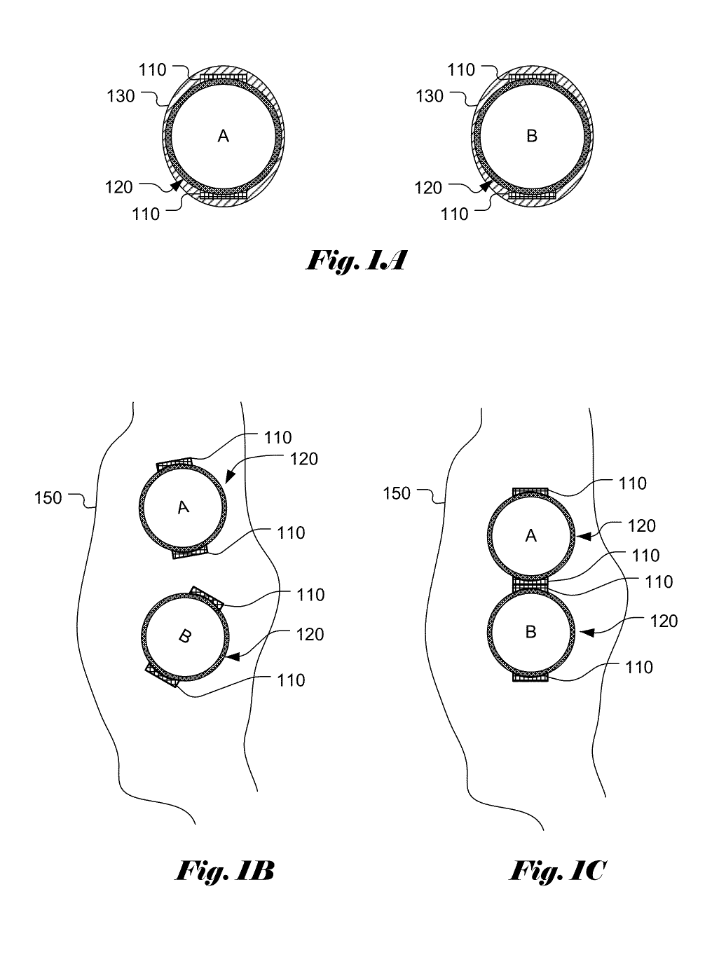

FIG. 1A illustrates one example to demonstrate the principle and shows schematically two endoscopic modules, A and B, prior to ingestion. The two modules may each perform the same or different functions, such as in vivo sensing or therapy delivery. Two or more modules may work in concert. For example, one module may provide illumination while the other contains a camera that captures images of the illuminated scene. Each module is of a size and shape that may be swallowed without difficulty. A spherical capsule should have diameter less than about 12 mm to permit easy swallowing. The hardware necessary to perform these functions is typically enclosed within a water-tight housing, although functional hardware, such as electrodes for transmission of electrical signals through the body, ports, or robotic appendages for collecting biopsies or delivering drugs, may also penetrate or form part of the housing. Each module has a means of connection to other modules. These connectors are shown schematically as plates 110 attached to the outside of the housing. The detailed form of the connector will vary. If the connector provides a force-exerting field, such as a magnet, it may reside inside the housing rather than outside.

A module should have a smooth surface when it is swallowed. A coating or outer capsule housing 130 may be applied to the capsule 120 as shown in FIG. 1A that dissolves in the GI tract. The coating or outer housing 130 may be made of polymers, polysaccharides, plasticizers, methyl cellulose, gelatin, sugar, or other materials. The coating may be enteric and designed to break down in the small bowel or colon rather than the stomach. If the connector resides inside the housing rather than outside, the coating may not be needed.

The two modules are swallowed in succession. Typically they will reside in the stomach for a period of time ranging from several minutes to 1.5 hours, and during this time they will tumble about and knock together. FIG. 1B shows the two modules in an internal organ 150 such as the stomach, where the two modules are separated. When the connector on one module touches, in the course of random motion, the connector of another module, the two will join together, as shown in FIG. 1C with a holding force that depends on the connection mechanism. The two joined modules then move through the GI tract together. For two modules to join, only one connector per module is required. However, the modules may have more connectors—two are shown in FIG. 1—so that the probability that a connection will fail to form is reduced. The modules need not necessarily join in the stomach. If the system is intended to operate in the colon, they could join in the small bowel or even in the cecum.

The connectors may also include electrical conductors for the transmission of power or electrical signals from one module to another. The holding force could be provided mechanically, by a latching mechanism for example. The attraction between a magnet in one module to a ferromagnetic component or to another magnet in another module may provide a holding force. Alternatively, the holding force could come from a chemical bonding between substances on one connector with substances on another connector. The holding force could also be provided by a combination of various means mentioned above. For example, while a mechanical means is used, magnetic means may also be used additionally to cause the modules to join easily. A connector system must provide sufficient holding force so that the connection is not easily broken by the forces exerted by the bowel (e.g. by peristalsis) on the system, especially as it passes through the pylorus and ileal-cecal valve.

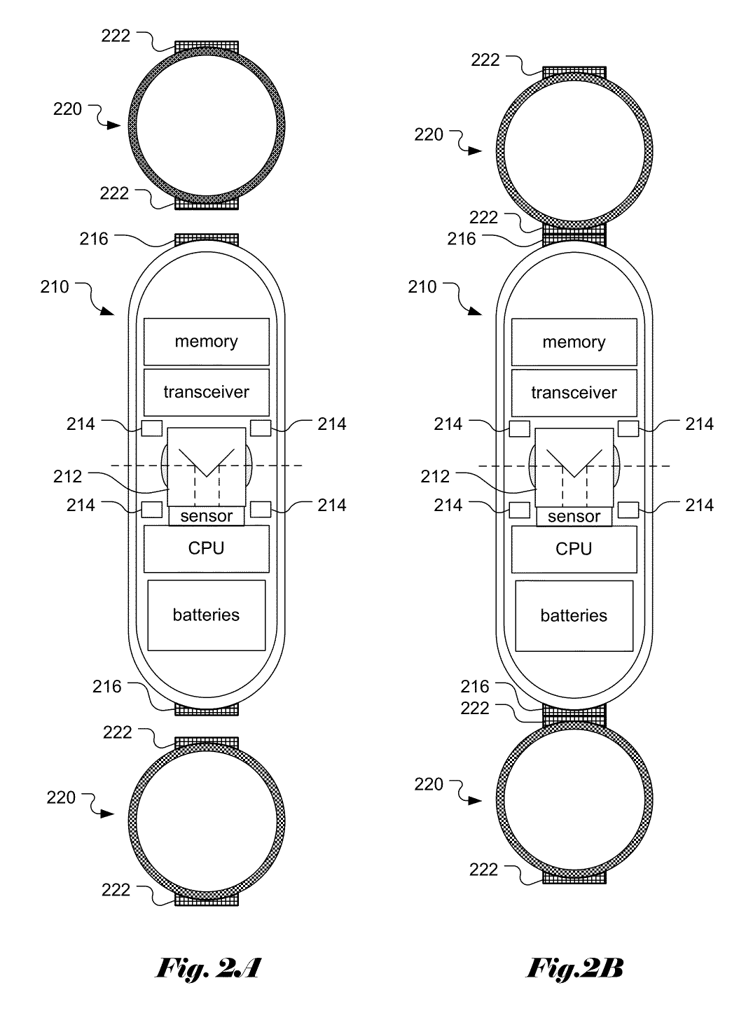

FIG. 2 illustrates two examples of a primary module connecting to one or more secondary modules. The primary system may be similar to legacy capsules with one or more batteries for power, one or more cameras (including image sensors and lenses), illumination light sources, a central processing unit (CPU), and a memory for archival storage and/or a transceiver for transmitting data to and receiving control signals from a remote base station outside the patient's body. FIGS. 2A and 2B illustrate a primary module 210 with camera objectives 212 located in the center of capsule facing the capsule sides. A capsule with four such side-facing objectives with optical axes spaced by 90° has been previously disclosed by CapsoVision. Multiple light sources 214 are shown inside the housing of the primary module 210. While same reference number 216 is used to indicate the connectors of primary module 210, the two connectors may be different. Furthermore, two connectors 216 are shown at the elongated ends of the primary module 210. Two connectors 222 are also shown for each secondary module 220. Again, while same reference number 222 is used to indicate the connectors of secondary module 220, the two connectors may be different. FIG. 2A illustrates a scenario that two secondary modules 220 are separated from primary module 210. FIG. 2B illustrates two secondary modules 220 connected with primary module 210. FIGS. 2C and 2D illustrate primary module 230 with a single camera facing one end. A connector 236 for attaching a secondary module 240 is located at the other end. Light sources 234 inside the primary module 230 are also shown. Two connectors 242 are also shown for secondary module 240. While same reference number 242 is used to indicate the connectors of secondary module 240, the two connectors may be different. The two modules in FIG. 2C are separated and the two modules in FIG. 2D are connected.

The secondary modules are shown as empty spheres, although they could take a variety of shapes and need not be empty. Once attached to the primary module as shown in FIGS. 2B and 2D, an assembly is formed. The secondary modules may contain hardware (not shown), including power supplies, system controls, memories, transceivers, cameras, light sources, propulsion devices, and sensors. Sensors could include optical sensors such as multi-spectral imagers or microscopes or chemical sensors such as pH sensors or bio-assay sensors. The secondary-module light sources could include visible or other wavelengths of light for image illumination or UV sources to excite fluorescence for fluorescence imaging. The secondary modules may instead be devoid of electronic hardware and provide a mechanical function when attached to the primary module.

A capsule endoscope assembly can move through the bowel more rapidly and with greater orientation stability than a traditional endoscopic capsule. In one embodiment, the two secondary modules 220 shown in FIGS. 2A and 2B are empty orbs. By attaching to either end of the primary module, they extend its length. An assembly that is longer than the colon lumen diameter will not easily tumble and will tend to align longitudinally with the colon. The secondary modules located at the primary module ends do not obstruct the primary module cameras' fields of view if the cameras are located in the middle of the module and face the sides, as shown in FIGS. 2A and 2B. The cameras face the mucosal surfaces of the organ. Four cameras together may capture a panoramic image of the mucosa. Alternatively, a panoramic imaging system may only have a single objective. The assembly moves through the colon with limited pitching and tilting, resulting in a more stable video with can be reviewed more quickly at higher frame rate with less fatigue than a video in which the camera is pitching and tilting.

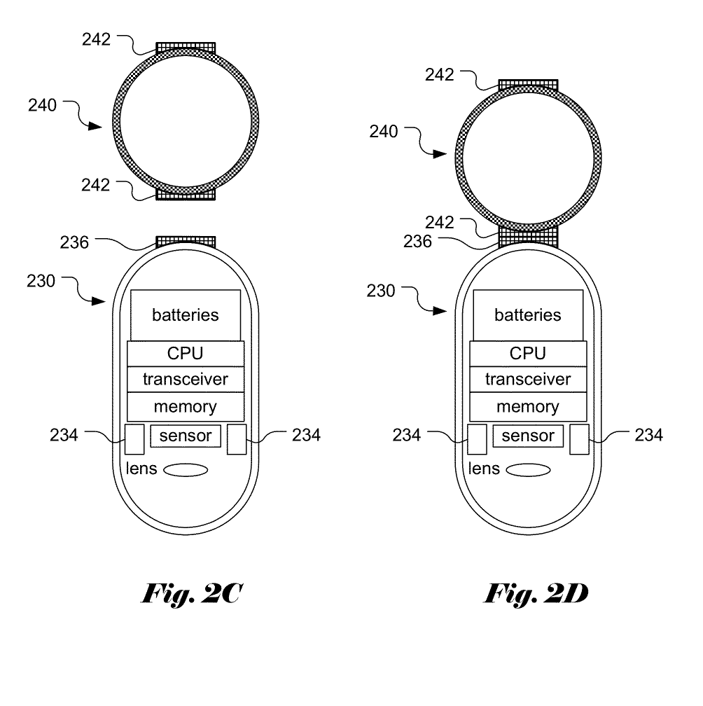

FIG. 3 shows two autonomous capsules 310 and 320 that are joined by connectors (316 and 326), thereby fixing their relative orientation inside the body. Each capsule has a single camera facing one end of the capsule. Each capsule also includes multiple light sources (314 and 324). The pair provides both a forward and backward view of the colon or other organ. Legacy capsules exist with a forward and backward facing camera in a single capsule. The capsule pair has twice the battery capacity as a single capsule, which enables higher frame rate and/or image resolution than a single capsule with two cameras and a single set of batteries. The increased length of the pair, compared to a single capsule, helps to keep it aligned to the longitudinal axis of the bowel. If the two capsules transmit by radio, they must avoid interference, for example, by using separate frequency channels. If they store data on board, interference is not an issue.

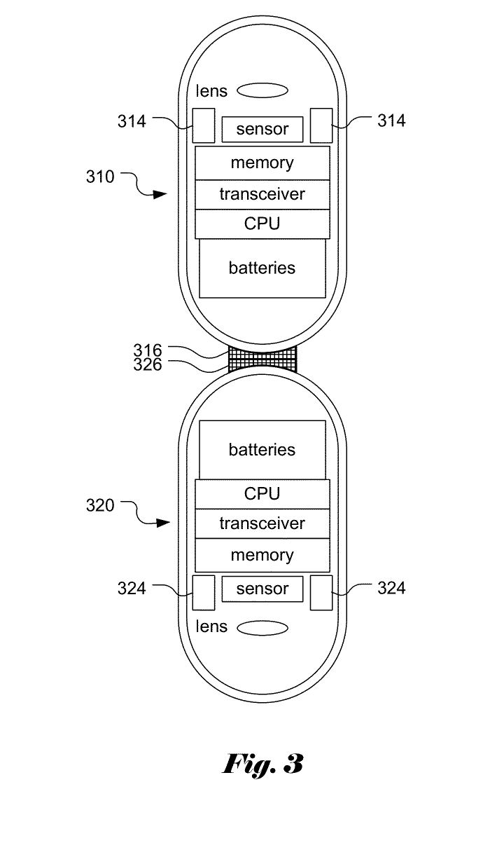

FIG. 4 shows a primary capsule module 410 and multiple secondary modules 420 in the stomach 430 with hook-and-loop connectors, which are shown schematically. Velcro is a common brand for such connection systems. The hooks are generally plastic and formed by a molding process. The loops often comprise a woven fabric with polymer fiber loops. Sections of hook or loop material could be cut from a sheet and affixed to portions of the modules with adhesive, Radio Frequency (RF) Welding, or other means. Alternatively, hooks could be molded directly onto a capsule housing. Loops could be attached to the housing directly, using adhesive coating, or by other means. In FIG. 4, hooks 412 are attached to primary module 410 and loops 422 are attached to secondary modules 420. However, loops may also be attached to primary module 410 while hooks are attached to secondary modules 420.

The hooks and loops are generally smaller and more densely spaced than those shown in FIG. 4. In fact, fields of small interlocking structures can be microscopic. The structures on both bonding surfaces may be hooks, rather than hooks on one surface and loops on another. Microscopic hooks have been fabricated on silicon substrates, for example. One form of proposed “nanohooks” would comprise hooks formed from carbon nanotubes. The bonding force achieved when nanohooks attached to one surface interlock with nanohooks attached to another surface is estimated to be 30 times stronger than conventional epoxy. U.S. Pat. No. 7,181,811 describes interlocking hooks forming a micro-fastening system and their method of manufacture.

The hooks and/or loops must be soft enough or small enough so as not to damage mucosal surfaces they contact. Additionally, the capsule surface must be smooth enough to allow easy swallowing and passage through the GI tract. Smaller hook and loop structures should generally be safer than larger ones. In order to provide a smooth surface for comfortable swallowing, the capsule modules can be coated in a manner similar to a drug tablet, covering the structures. Coating materials may be or may not be enteric and may include materials such as polymers, polysaccharides, plasticizers, methyl cellulose, gelatin, sugar, and pigments. Methacrylic acid co-polymer type C is an example of an enteric polymer. When the coating dissolves inside the body, the hooks and loops are exposed and connections between modules can occur.

FIG. 4 shows a main (primary) capsule (410) with hooks on either end and three satellite (secondary) modules (420) with loops covering their entire surface. When hook-loop pair is used, the use of hooks and loops may be reversed. For example, the main (primary) capsule (410) may have loops on both ends and three satellite (secondary) modules (420) may have hooks covering their entire surface. For convenience, the term “hook/loop” is used to indicate the alternative use of hook and loop. The corresponding connector is indicated by “loop/hook”. The modules are shown in the stomach after they have been swallowed. In this example, with spherical satellites, the rotational orientation of the satellites is unimportant and the probability of attaching to the main capsule increases with the area of the loop coverage. If the diameter of the spheres is less than or equal to the diameter of the primary capsules, then they will not increase the width of the assembly, regardless of orientation. Because of the limited hook area on the main capsule in two locations, only two satellite modules can connect to the main module. Extra satellite modules may be swallowed to increase the probability that one will attach to each end of the capsule before it leaves the stomach.

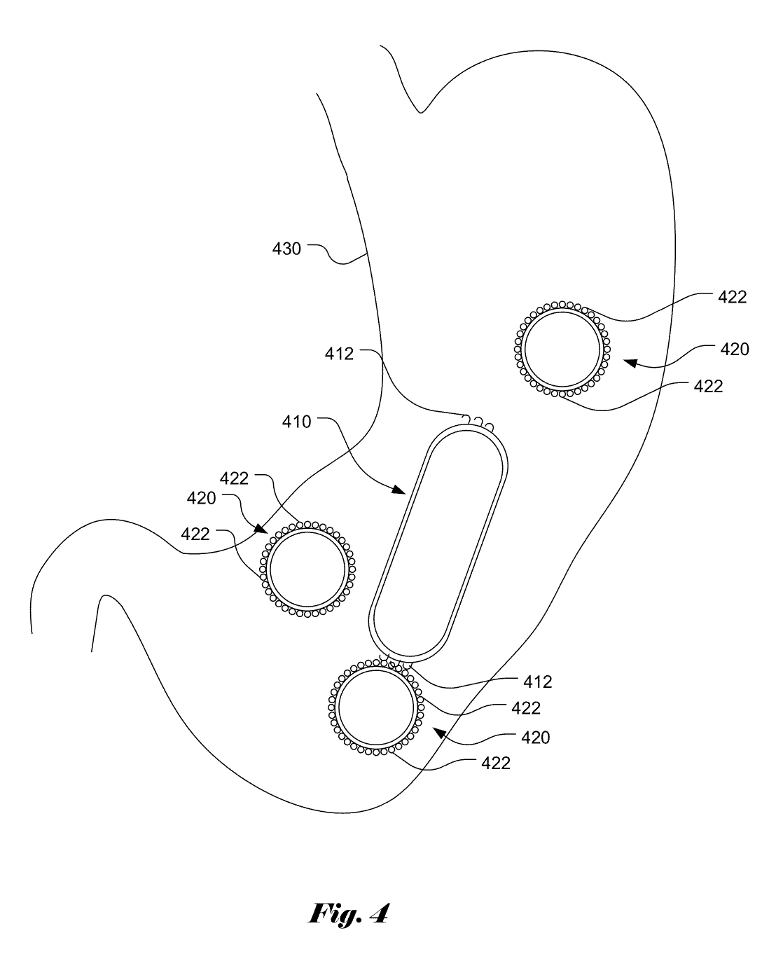

Capsule modules can be connected by the attraction of magnets. Each of two modules may have a connector formed from or containing one or more magnets, or one module may have a magnet and the other an unmagnetized ferromagnetic material, such as steel, or a ferromagnetic material, such as ferrite. For convenience, the piece that interacts with the magnet of a connector is called an “interaction piece” in this disclosure. The interaction piece may be a magnet or any unmagnetized ferromagnetic or ferrimagnetic material. Magnetic connectors attract or repel, depending on the relative orientation of their polarities. Ferromagnetic or ferrimagnetic connectors are attracted to magnets of any polarity but are not attracted to each other. These properties allow the design of groups of capsule modules that will assemble by themselves in predetermined configurations. FIG. 5 illustrates a few examples. In 5A, the connectors 512 and 514 in modules A and B attract because their polarity is opposite; one is oriented with the south pole (S) facing out (type-v), and the other with the north pole (N) facing out (type-u). The rotational orientation of A relative to B does not affect the attraction if the connectors have a symmetrical shape. Two modules with type-u connectors will not connect since the connectors will repel. In 5B, the modules A and B are identical with magnetic connectors 522 and 524 respectively that have polarities oriented tangential to the capsule surface (type-w). The connectors attract when the modules align with opposing polarities as shown in FIG. 5B. In this case, the rotational alignment of A to B is fixed once the connectors mate, and this rotational alignment property could be significant for some applications. In 5C, one module with connector type-u (532) is attracted to a module with connector type-x (534), which has an unmagnetized ferromagnetic or ferrimagnetic connector. Two connectors of type-x will not attract. In FIG. 5D, a module with type-w connector (542) connects to a module with type-x connector (544).

In the case of mechanical based connection using hooks-loops, the connectors need to be in physical contact before the connection can be made. The use of magnet may cause the capsule modules to connect when the two capsule modules are within a distance. The distance for two capsule modules to connect depends on the attraction force of the magnet and the interaction piece, physical characteristics of the capsule modules (such as the weight), and the environment surrounding the capsule modules (such as the liquid environment inside the stomach).

In the above examples, the magnets may be made from a variety of materials including rare earth metal alloys such as neodymium iron boron (NIB) or samarium cobalt, ferrites or other ceramics, or ferromagnetic metals. The magnets may be coated with inert metals such as nickel or gold. They may also be coated with polymers or epoxies. Coatings may provide biocompatibility and prevent the magnets, which may be brittle, from chipping. The magnets may be embedded in or enclosed within the housing of the capsule. Additional coatings may cover the magnets that dissolve in the GI tract. These coatings may provide a smoother shape to the capsule, as shown in FIG. 1A, to facilitate swallowing.

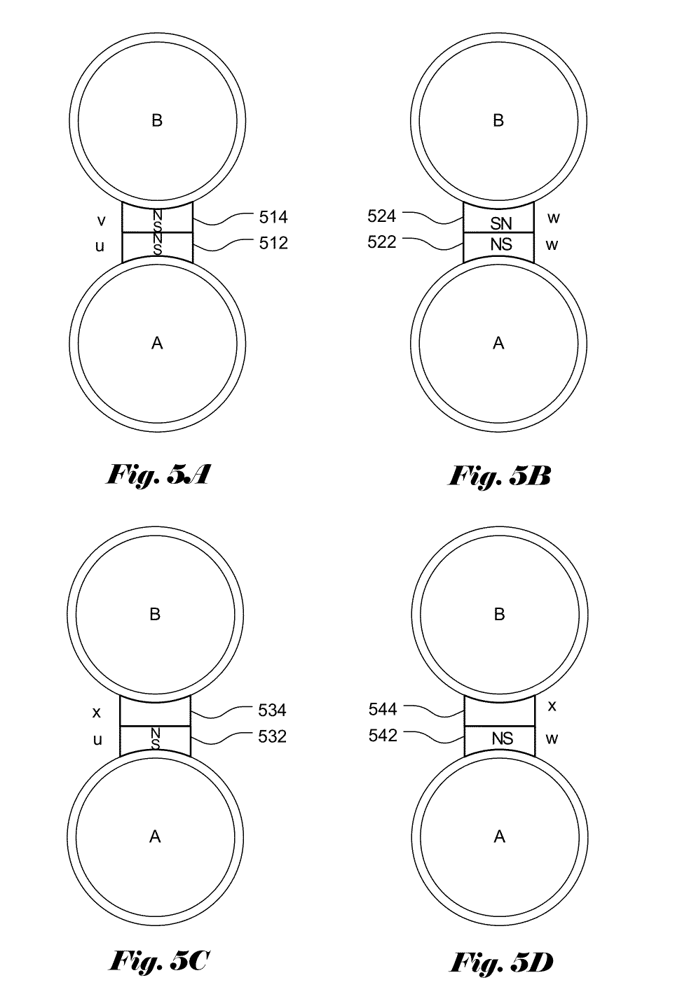

Magnets in the GI tract do pose potential hazards. If two magnets attract through an intervening layer of tissue, the tissue may become pinched and damaged. FIG. 6 shows two capsule modules 610 and 620 located in the small bowel 630. One is more distal along the bowel than the other, but they are close in three dimensional space. The magnets 612 and 622 are separated by at least twice the thickness of the bowel wall. The mutual attraction of the magnets exerts a force, and this force may damage the bowel wall if the force is too large at the minimum separation. The capsules may also become stuck in this position.

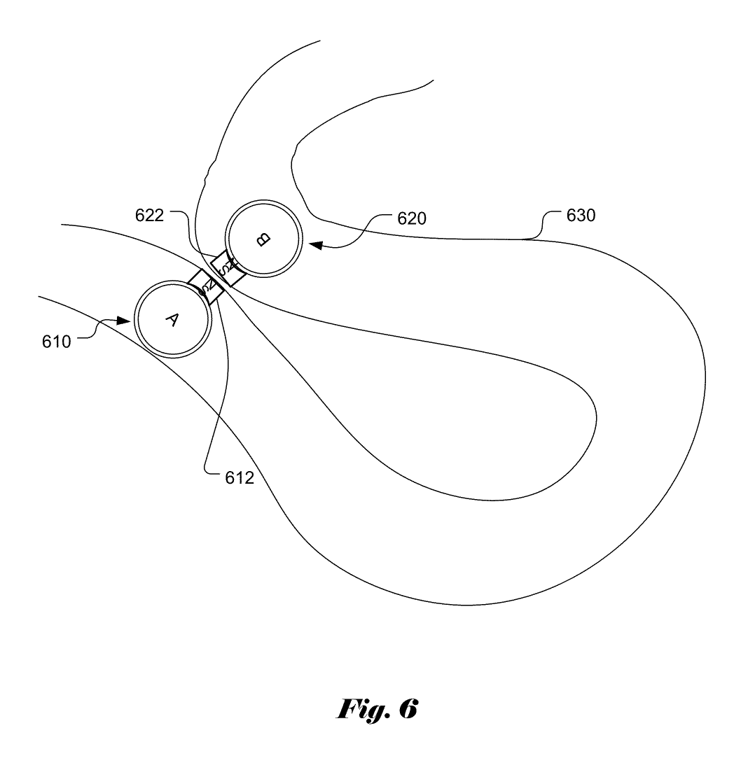

Coded magnets can be designed to produce short-range magnetic fields that produce attractive forces between magnets that are large at small separations and that fall off rapidly with increasing separation. Numerous patents have been assigned to Correlated Magnetics Research, Inc. covering the design, use, and manufacture of coded magnets (Polymagnets®). A coded magnet contains pixel-like magnetic domains (maxels), each of which is either of positive or negative polarity. An array of maxels produces an aggregate magnetic field whose properties are determined by the pattern, or code, of the pixel polarities. The attraction or repulsion of coded magnets depends on the correlation in the spatial domain between the two patterns. FIG. 7A schematically shows two coded magnets 710 and 720, one with Code A (710) and the other with Code B (720). The white spots represent positive polarity maxels and the shaded negative polarity maxels. The two coded magnets are attracted when they are aligned so that one code correlates to the inverse of the other. Two magnets with code A repel each other. In FIG. 7B the maxels are arranged in rings. Magnets 730 and 740 with code A and B are attracted, regardless of rotational orientation due to the symmetry of the patterns. Capsules can include connectors that contain coded magnets. If the codes are rotationally symmetric, then the connectors attract regardless of the rotational alignment of the capsules, which increases the chance that capsules will connect through random motion within an organ. If a particular rotational alignment is desired, for example to make an electrical connection between capsules, then codes without rotational symmetry can be used. The polarity patterns of code A magnet and code B magnet in FIG. 7A and FIG. 7B are complimentary. Accordingly, the pair of coded magnets are also called complimentary coded magnets.

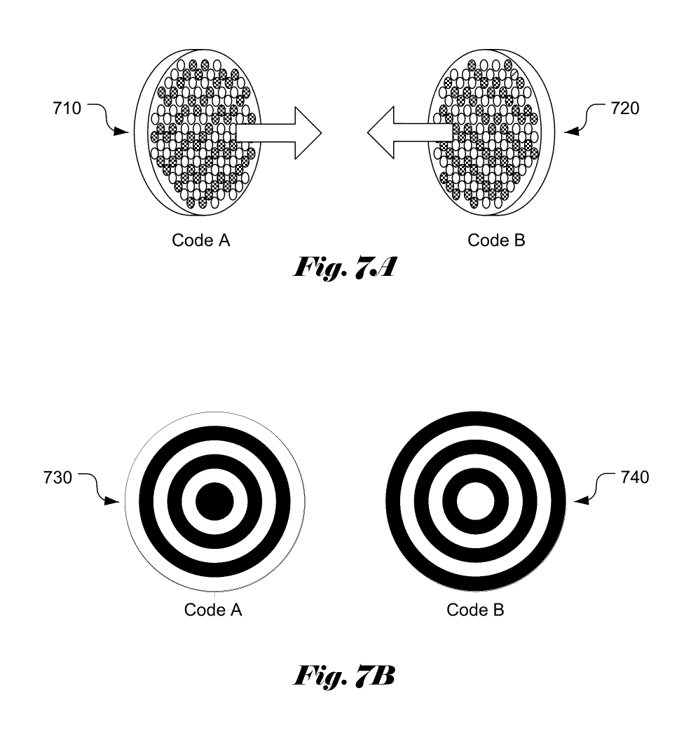

FIG. 8A shows a plot of the attraction force between magnetic connectors as a function of their axial separation. The solid line is for two coded magnets, and the dashed line is for two standard magnets with uniform magnetization. For zero separation the forces are equal, but the force between the coded magnets falls off much faster with separation. FIG. 8B shows two magnet connectors 812 and 822 of two respective modules 810 and 820 with an intervening layer of tissues of thickness X. If the force is at a safe level that will not damage or compress the tissue at separation X and X is the minimum tissue thickness that will be encountered during an in-vivo procedure, then the system is nonhazardous.

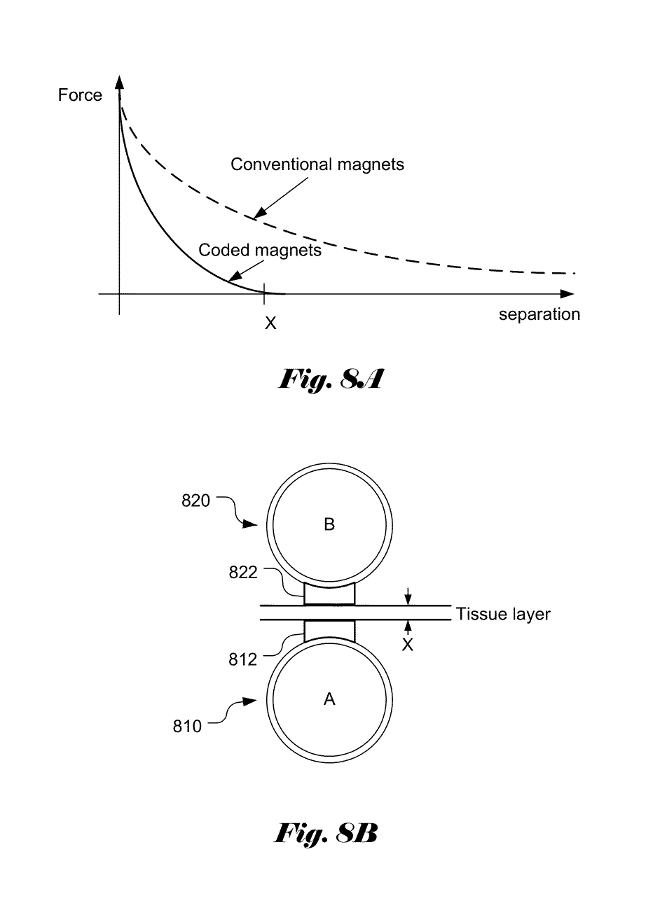

The stability of a composite capsule system in the colon increases as its length increases. However, beyond a certain length, the system may not easily pass through bends in the colon or small bowel. A system that articulates at one or more locations can provide stability while passing easily through a convoluted bowel. In FIG. 9, two capsules 910 and 920 have magnetic connectors 912 and 922 with domed surfaces facing the ends. The magnets may be standard magnets or coded magnets. The capsules are connected and have a relative tilt of θ. The magnets exert a tilting force to bring θ to zero, in the absence of other forces. However, θ can increase as the system passes through a bend in the GI tract.

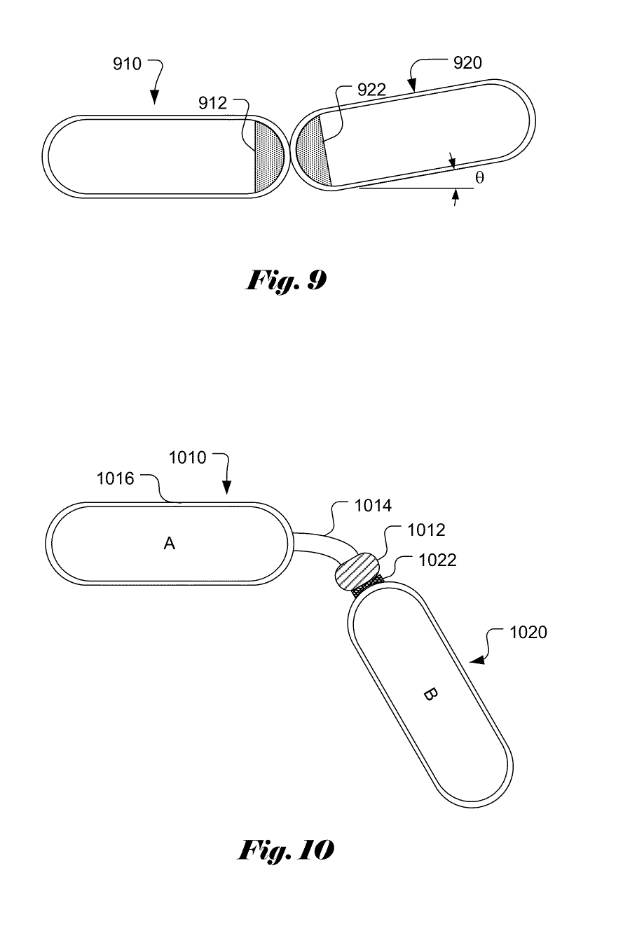

The connector on a capsule module may also have the ability to pivot. In FIG. 10, capsule A (1010) has a flexible member 1014 between the main housing 1016 and connector A (1012). Capsule module B (1020) has a connector 1022 that does not pivot. After connector A (1012) and connector B (1022) mate, the assembly articulates by the bending of the flexible member 1014. The flexible member 1014 may be semirigid so that some force is required to bend it. It may alternatively be nonrigid and serve as a tether. Such a tether might initially be coiled or bunched up inside or against one capsule and later deployed. The flexible member, which may be a tether, may be contained within a soluble coating or outer housing during swallowing. Connectors may also be designed to pivot relative to the capsule body by including hinges, bearings, pins, or variety of other similar rotating or flexing joint mechanisms.

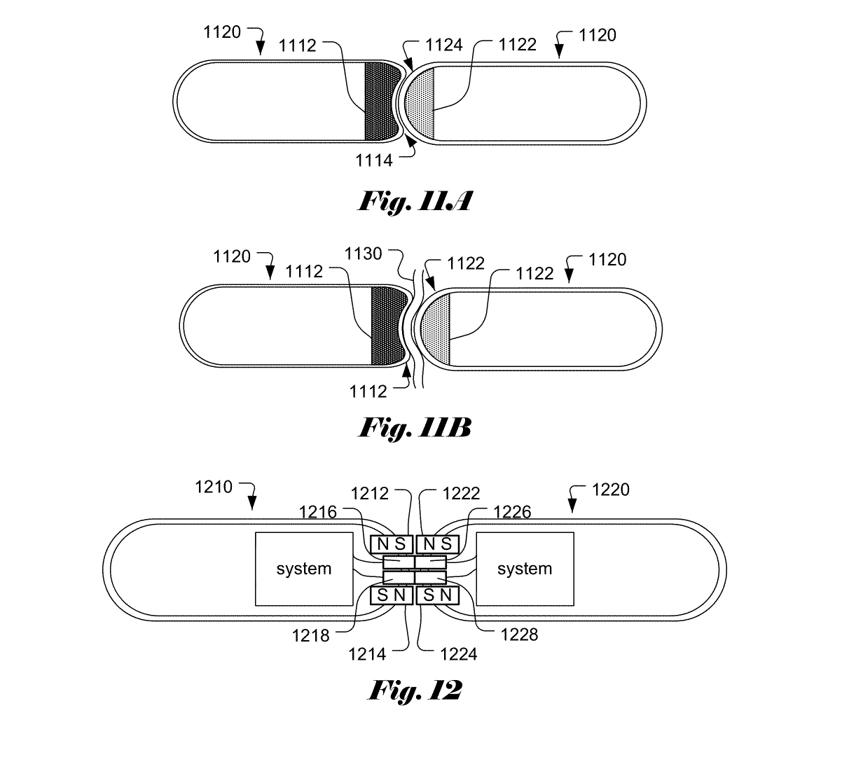

In FIG. 11A, one capsule (1120) has a convex surface (1124) and the other (1110) has a concave surface (1114) at its tip. Each capsule also has a connector at the tip. In this example, they are magnetic connectors 1112 and 1122. The convex surface of one capsule connects to the concave surface of the other and the curvatures of the two surfaces are approximately equal. The contact force is distributed over a larger area than for the configuration of FIG. 9, and the pressure is reduced. As a result, the pressure on any intervening tissue is reduced. FIG. 11B shows the two capsule sections with an intervening tissue 1130. If the left side of the tissue matches the curvature of the concave surface, the right side of the tissue has a smaller radius of curvature, assuming the tissue maintains a constant thickness. As a result, the capsules maintain a separation greater than the thickness of the tissue. The attractive force between the two capsules is thus less than the case for the two capsules with flat magnetic connectors and intervening tissue shown in FIG. 8.

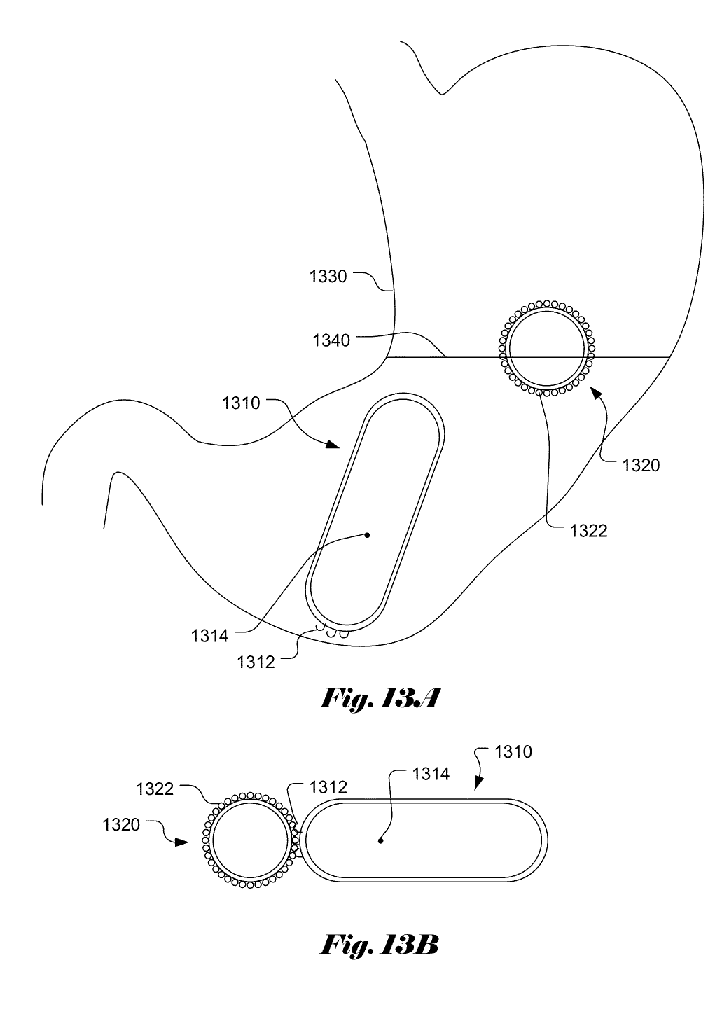

FIG. 12 illustrates an electrical connection between two capsules 1210 and 1220. The capsules are aligned and joined by a connector system comprising two magnets (1212/1214 and 1222/1224), one per capsule. Additional magnets or coded magnets could also be used. Each capsule has a pair of terminals (1216/1218 and 1226/1228) penetrating the housing. Helped with the pulling and holding force of the magnets attraction, the terminals make contact. In FIG. 12, two pairs of magnets are used and configured to ensure that the two capsules will be mated to cause the two electrical terminals connected in a correct orientation. While the pair of magnet connectors also penetrates through the housings of the capsules as shown in FIG. 12, the pair of magnet connectors may also be disposed on the outside of the capsules housings, similar to the arrangement as shown in FIG. 3. Alternatively, the pair of magnets can be inside the capsules, and the housings cover around the magnets on the connecting side. Therefore, only the electrical terminals penetrate through the housings respectively.

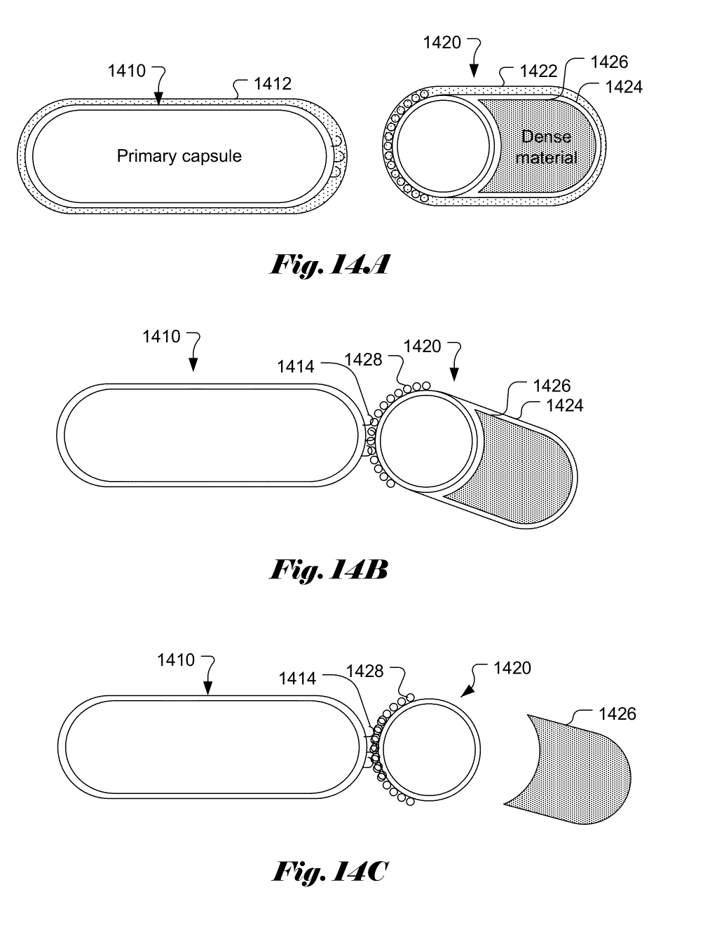

FIG. 13 shows a primary capsule (1310) of specific gravity greater than one and a secondary capsule (1320) with specific gravity less than one in an organ such as stomach 1330. The secondary capsule (1320) will float above liquid level 1340 in the organ such as the stomach as shown in FIG. 13A. When the two capsules join, the assembly will have an intermediate specific gravity. The primary capsule may have a center of mass 1314 that is closer to one end than the other. It has hooks 1312 (or another connector type) at this end only so that when the less dense secondary capsule (1320) attaches to primary capsule 1310, the ensemble will be balanced with the center of mass (1314) near the center of the assembly as shown in FIG. 13B. The secondary capsule (1320) is shown as a sphere covered with hooks 1322. Other shapes or connector systems are possible as well.

Within the stomach, the high-specific gravity primary capsule will tend to sink with the heavier end bearing the connector tilted down, while the secondary capsule will tend to float. Although the motor activity of the stomach will jostle both capsules and they may collide with the correct orientation to connect, a significant risk exists that they will not.

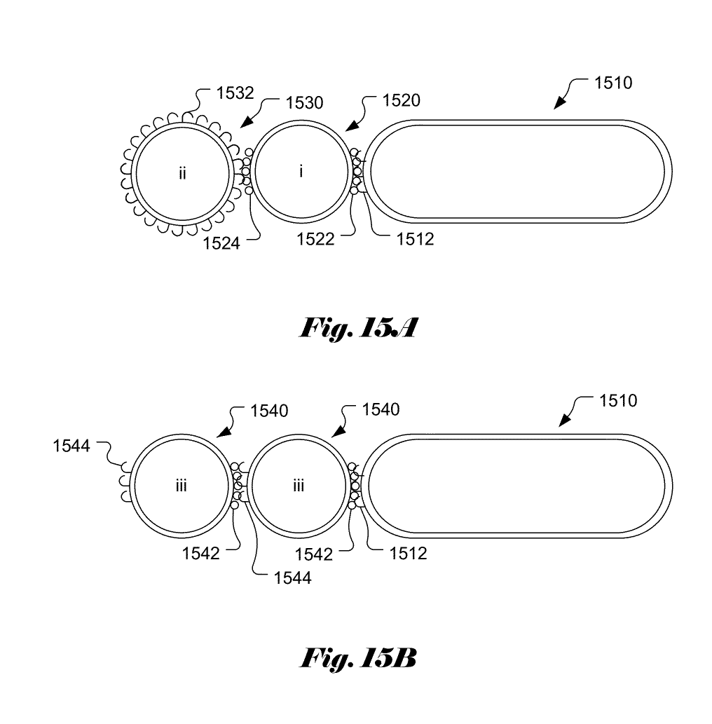

In order to increase the probability of forming a connection, the specific gravity of the secondary capsule can be temporarily increased to a value greater than one so that it sinks in the stomach fluid. FIG. 14A shows a primary capsule (1410) and a secondary capsule (1420) prior to swallowing or just after swallowing. Each capsule has a coating (1412 and 1422) to cover the connectors that dissolves rapidly after swallowing. After the coating dissolves, the connectors (1414 and 1428) for the primary capsule and the secondary capsule will be exposed. The secondary capsule is attached to a dense material (1426) that is encapsulated in a “shell” (1424). The shell is enteric and only dissolves after passage from the stomach. The dense material may comprise powder or granules or it may be a single solid mass. The material could be a biocompatible metal such as stainless steel or it could be a ceramic such as titanium dioxide or zirconium dioxide. The combination of the secondary capsule, the shell, and the dense material has an overall specific gravity greater than one. Thus, it will sink to the bottom of the stomach so that it can more readily connect to the primary capsule, which also will sink as shown in FIG. 14B. After the assembly leaves the stomach, the shell dissolves and the dense material (1426) separates from the secondary capsule(1420) as shown in FIG. 14C. If it is a powder, it will disperse and eventually be excreted. If it is a solid object, it will be passed from the GI tract along with the capsule assembly.

Instead of using a shell to hold the dense material to the secondary capsule, a solid dense object could be attached to the secondary capsule with an adhesive that dissolves in the small bowel or colon but not in the stomach. Such an adhesive could resist stomach acid but dissolve in the higher pH of the small bowel and colon. Alternatively, it could be attacked by bacteria residing in the small bowel or colon. A granular dense medium might be cemented together and attached to the secondary capsule with one or more such adhesives. The medium would then break apart after passing from the stomach. A dense object might attach to the secondary capsule with a hook or other connecting mechanism that is made of a material that breaks apart after leaving the stomach due to the change in pH or attack by bacteria.

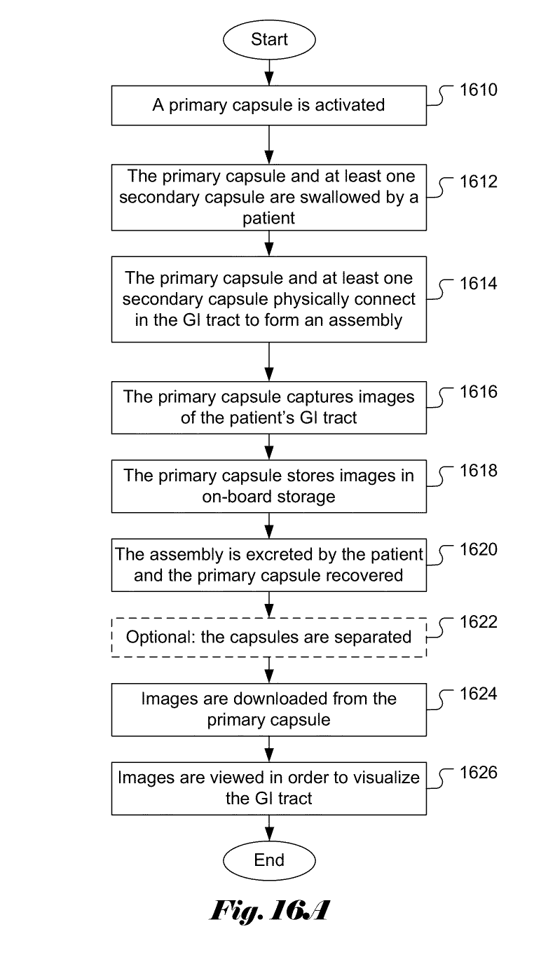

Chains of secondary capsules can be attached to the end of a primary capsule. In FIG. 15A, one type-i secondary capsule (1520) has loops 1522 and 1524 located at two poles. A type-ii second secondary capsule (1530) has hooks 1532 on its entire surface. The patient could swallow one primary capsule (1510), one type-i capsule of type, and one or more type-ii capsules, and the linear assembly of FIG. 15A should result. Alternatively, the patient could swallow the primary capsule and two or more type-iii capsules (1540) and the linear result shown in FIG. 15B should result. Type-iii capsules have hooks 1544 at one pole and loops 1542 at the other. A roughly linear arrangement can be important for minimizing the chance that the assembly will be unable to pass through the strictures and possible obstructions in the GI tract. As mentioned before, the use of hooks/loops on these modules can be reversed. Furthermore, instead of using both hooks and loops, hooks alone can be used on the modules for connection.

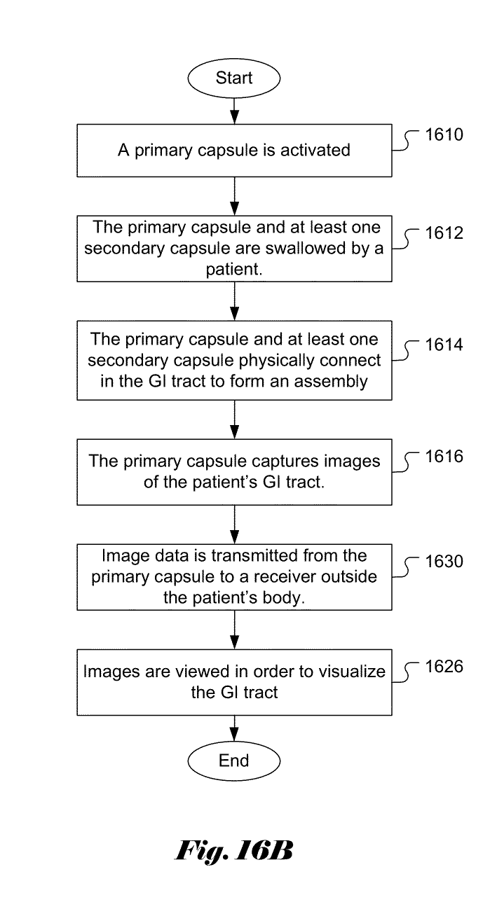

Usage scenarios of capsule endoscopy incorporating embodiment of the present invention are illustrated in FIG. 16A and FIG. 16B. FIG. 16A illustrates a flow of the events during the operation of the capsule endoscopy, where the capsule includes on-board storage. Prior to swallowing the primary capsule, it is activated as shown in step 1610—meaning that the internal system is connected to a power source such as internal batteries. A patient then swallows the primary capsule and at least one secondary capsule as shown in step 1620. Within the patient's GI tract, the primary capsule and at least one secondary capsule will form an assembly as shown step 1614. The primary capsule captures images while travelling through the patient's GI tract in step 1616 and stores the images in on-board storage in step 1618. After the assembly is excreted by the patient, the primary capsule is recovered as shown in step 1620. If the primary capsule is connected to any secondary capsule, the capsules may be separated in the optional step 1622. The images are downloaded from the primary capsule in step 1624 and are viewed in order to visualize the GI tract in step 1626.

FIG. 16B illustrates a flow of the events during the operation of the capsule endoscopy, where the capsule uses wireless transmission to send images to a receiver outside the patient's body. Step 1610 through step 1616 are the same as the case with on-board storage. For the capsule with wireless transmission, the image data is transmitted from the primary capsule to a receiver outside the patient's body as shown in step 160. The images are viewed in order to visualize the GI tract in step 1626.

The invention may be embodied in other specific forms without departing from its spirit or essential characteristics. The described examples are to be considered in all respects only as illustrative and not restrictive. Therefore, the scope of the invention is indicated by the appended claims rather than by the foregoing description. All changes which come within the meaning and range of equivalency of the claims are to be embraced within their scope.

1. An in-vivo capsule system, comprising:
a first primary capsule;
at least one second capsule, wherein said at least one second capsule corresponds to one or more other primary capsules, one or more secondary capsules, or any combination thereof; and
a self-connection means for connecting the first primary capsule and said at least one second capsule by providing holding force when the first primary capsule and said at least one second capsule are in contact or within a distance in GI tract.

2. The in-vivo capsule system of claim 1, wherein the self-connection means comprises an interlocking means disposed on the first primary capsule and said at least one second capsule.

3. The in-vivo capsule system of claim 2, wherein the interlocking means corresponds to hooks disposed on one connecting side and loops disposed on the other connecting side, or hooks disposed on both connecting sides.

4. The in-vivo capsule system of claim 2, wherein at least a part of the first primary capsule or said at least one second capsule is coated with a coating material that dissolves in the GI tract.

5. The in-vivo capsule system of claim 4, wherein the coating material is selected from a group comprising polymers, polysaccharides, plasticizers, methyl cellulose, gelatin, and sugar.

6. The in-vivo capsule system of claim 4, wherein said at least one second capsule corresponds to one secondary capsule, the first primary capsule has a first specific gravity larger than one and said one secondary capsule has a second specific gravity smaller than one.

7. The in-vivo capsule system of claim 6, wherein said one secondary capsule and a dense material are enclosed in a shell, overall specific gravity of said one secondary capsule and the dense material in the shell is greater than one to cause said one secondary capsule and the dense material in the shell to sink in stomach, and the shell dissolves when the in-vivo capsule system leaves the stomach.

8. The in-vivo capsule system of claim 1, wherein the self-connection means corresponds to a first magnet and an interaction piece affixed to two corresponding capsules respectively, and the interaction piece corresponds to a second magnet, a ferromagnetic component, or a ferrimagnetic component.

9. The in-vivo capsule system of claim 8, wherein the first magnet and the interaction piece are disposed inside or outside the two corresponding capsules, or reside inside housings of the two corresponding capsules.

10. The in-vivo capsule system of claim 8, wherein the first magnet and the interaction piece are configured to penetrate through housings of the two corresponding capsules while maintaining the housings of the two corresponding capsules sealed.

11. The in-vivo capsule system of claim 8, wherein the first magnet and the interaction piece are complimentary coded magnets, the complimentary coded magnets are configured to cause extraction force below a safe level when separation between the complimentary coded magnets is greater than a threshold, and the threshold corresponds to tissue thickness.

12. The in-vivo capsule system of claim 8, wherein the first magnet and the interaction piece are outside the two corresponding capsules, and one of the first magnet and the interaction piece is fixedly attached to one of the two corresponding capsules through a flexible member.

13. The in-vivo capsule system of claim 12, wherein the flexible member is semi-rigid.

14. The in-vivo capsule system of claim 13, wherein the flexible member is a non-rigid flexible member, and the non-rigid flexible member is served as a tether that is coiled or bunched inside or again said one of the two corresponding capsules.

15. The in-vivo capsule system of claim 8, wherein the first magnet and the interaction piece are formed into mating concave-convex contact surfaces.

16. The in-vivo capsule system of claim 8, wherein the self-connection means further comprises electrical connection between said two corresponding capsules.

17. The in-vivo capsule system of claim 1, wherein the first primary capsule comprises:
an image sensor;
an optical subsystem to form an image onto the image sensor;
illumination light sources;
a processing unit for system control and processing; and
on-board storage to store captured images or a wireless module to transmit the captured images.

18. The in-vivo capsule system of claim 1, wherein said at least one second capsule corresponds to one other primary capsule, one secondary capsule, or two secondary capsules.

19. The in-vivo capsule system of claim 18, wherein the self-connection means comprises an interlocking means disposed on the first primary capsule and said two secondary capsules, the interlocking means corresponds to hooks and loops, the hooks/loops are disposed on the first primary capsule and one of said two secondary capsules, and the loops/hooks are disposed on other of said two secondary capsules.

20. The in-vivo capsule system of claim 18, wherein the self-connection means comprises an interlocking means disposed on the first primary capsule and said two secondary capsules, the interlocking means corresponds to hooks and loops, the hooks/loops are disposed on the first primary capsule and first ends of said two secondary capsules, and the loops/hooks are disposed on second ends of said two secondary capsules.

21. The in-vivo capsule system of claim 1, wherein the first primary capsule comprises a first connector on a first end of the first primary capsule and a first camera facing opposite to the first end, said at least one second capsule corresponds to a second primary capsule, the second primary capsule comprises a second connector on a second end of the second primary capsule and a second camera facing opposite to the second end, and the first connector and the second connector connect when the first primary capsule and the second primary capsule are in contact in the GI tract.

22. The in-vivo capsule system of claim 1 wherein a second housing of the second capsule is able to pivot relative to a first housing of the first primary capsule when an external force is supplied, and the holding force returns the second housing of the second capsule to an original position when the external force is removed.

23. A method of imaging human GI track using an in-vivo capsule system comprising a primary capsule and a secondary capsule, the method comprising:
activating the primary capsule, wherein the primary capsule includes a camera;
swallowing the primary capsule by a patient;
swallowing the secondary capsule by the patient, wherein the in-vivo capsule system provides holding force for connecting the primary capsule and the secondary capsule when the primary capsule and the secondary capsule are in contact in GI tract;
capturing images of the GI tract using the camera; and
storing capture images in on-board storage inside the primary capsule or transmitting the captured images wirelessly to a receiving device outside patient's body.

24. The method of claim 23, if the captured images are stored in the on-board storage, further comprising:
recovering the primary capsule when the in-vivo capsule system is excreted by the patient; and
downloading the captured images from the primary capsule.
